(19) United States
(12) Patent Application Publication
(10) Pub. No.: US 2023/0266294 A1
(43) Pub. Date: Aug. 24, 2023

(54) SYSTEMS AND METHODS FOR MONITORING WATER QUALITY IN A BUILDING

(71) Applicant: David James SCOTT, Toronto (CA)

(72) Inventor: David James SCOTT, Toronto (CA)

(21) Appl. No.: 17/675,204

(22) Filed: Feb. 18, 2022

(51) Int. Cl. G01N 33/18; G01D 21/02

(52) U.S. Cl. CPC G01N 33/182; G01D 21/02

(57) ABSTRACT

A method for operating a water distribution system of a building is disclosed. The method includes: connecting a first sensor assembly to the water distribution system at a first connection point such that influent water from a water treatment system flows at least partially through the first sensor assembly; connecting one or more second sensor assemblies at second connection points that are downstream of the first sensor assembly in the water distribution system; obtaining, via sensors of the first and second sensor assemblies, first sensor data and second sensor data, respectively; detecting changes in at least one of chlorine concentration, temperature, or pressure of water between the first connection point and the one or more second connection points based on the first and second sensor data; responsive to determining that the detected changes satisfy defined criteria, generating a message identifying the detected changes and a first system operation for performing in connection with the water quality monitoring system; and transmitting the generated message to a remote computing device associated with the water quality monitoring system.

### TECHNICAL FIELD

The present disclosure relates to water supply networks and, in particular, to systems and methods for monitoring quality of water that is distributed in a building.

### BACKGROUND

Water quality monitoring and control are critical to maintaining the integrity of a water distribution system and the health of a property's water users. Degradations in water quality can be caused by microorganism growth, nitrification, and internal corrosion of pipes, among other factors. Poor maintenance or operation of a water system can expose people to risk of contracting various diseases, such as Legionnaires' disease or non-tuberculosis mycobacteria (NTM).

### BRIEF DESCRIPTION OF DRAWINGS

Reference will now be made, by way of example, to the accompanying drawings which show example embodiments of the present application and in which:

FIG. 1 is a schematic diagram illustrating an operating environment of an example embodiment;

FIG. 2A is high-level schematic diagram of an example computing device;

FIG. 2B shows a simplified organization of software components stored in a memory of the example computing device of FIG. 2A;

FIG. 3 shows, in flowchart form, an example method for operating a water quality monitoring system of a building;

FIG. 4 shows, in flowchart form, another example method for operating a water quality monitoring system of a building; and

FIG. 5 shows, in flowchart form, an example method of providing, on a client device, a user interface for controlling a water quality monitoring system.

Like reference numerals are used in the drawings to denote like elements and features.

### DETAILED DESCRIPTION OF EXAMPLE EMBODIMENTS

In an aspect, the present disclosure describes a system for monitoring water quality in a water distribution system of a building or property. The system includes a first sensor assembly and one or more second sensor assemblies. Each of the first and second sensor assemblies includes: a chlorine sensor for detecting a concentration of chlorine in water; a temperature sensor; and a pressure sensor. The first sensor assembly is fluidly connected to the water distribution system at a first connection point such that influent water from a water treatment and supply system flows at least partially through the first sensor assembly and the one or more second sensor assemblies are disposed downstream of the first sensor assembly in the water distribution system at one or more second connection points. The system also includes a processor and a memory coupled to the processor. The memory stores computer-executable instructions that, when executed by the processor, cause the processor to: obtain first sensor data via sensors of the first sensor assembly; obtain second sensor data via sensors of the one or more second sensor assemblies; detect changes in at least one of chlorine concentration, temperature, or pressure of water between the first connection point and the one or more second connection points based on the first and second sensor data; responsive to determining that the detected changes satisfy defined criteria, generate a message identifying the detected changes and a first system operation for performing in connection with the system; and transmit the generated message to a remote computing device associated with the system.

In some implementations, the remote computing device may be a valve control system that is operable to control valves for flushing water from pipes of the water distribution system and the message may include control signals for varying a valve control system based on the detected changes in the at least one of chlorine concentration, temperature, or pressure of water in the water distribution system.

In some implementations, the valves may be controlled to flush water from the pipes of the water distribution system responsive to detection of one or more trigger conditions relating to the first and second sensor data.

In some implementations, each of the first and second sensor assemblies may include at least one of: flow control valves; pressure regulator valves; a variable-area flowmeter; a turbidimeter; or one or more drain pipes.

In some implementations, the one or more second sensor assemblies may be disposed at one or more distal locations along the water distribution system.

In some implementations, detecting the changes may include: determining an amount of change in the at least one of chlorine concentration, temperature, or pressure of water between the first and second connection points; and comparing the amount of change to one or more defined threshold values.

In some implementations, the computer-executable instructions, when executed by the processor, may further cause the processor to generate historical trends data based on sensor data obtained via sensors of the first and second sensor assemblies, and the control signals may be generated based on the detected changes and the historical trends data.

In some implementations, the computer-executable instructions, when executed by the processor, may further cause the processor to: determine that at least one of the first sensor data or the second sensor data satisfies a defined trigger condition; and in response to the determining: generate a notification message identifying the trigger condition; and transmit the notification message to a client device associated with the water distribution system.

In some implementations, the trigger condition may be associated with a threshold value and the notification message may include an indication of a suggested action for rectifying a water quality issue associated with the threshold value.

In some implementations, the computer-executable instructions, when executed by the processor, may further cause the processor to receive, via the client device, user input including definitions of the trigger condition.

In some implementations, the computer-executable instructions, when executed by the processor, may further cause the processor to: generate display data including graphical representations of sensor data associated with the first and second sensor assemblies; and transmit the display data to a client device associated with the water distribution system.

In another aspect, the present disclosure describes a method for operating a water quality monitoring system of a building. The method includes: connecting a first sensor assembly to the water distribution system at a first connection point such that influent water from a water treatment and supply system flows at least partially through the first sensor assembly; connecting one or more second sensor assemblies at second connection points that are downstream of the first sensor assembly in the water distribution system; obtaining, via sensors of the first and second sensor assemblies, first sensor data and second sensor data, respectively; detecting changes in at least one of chlorine concentration, temperature, or pressure of water between the first connection point and the one or more second connection points based on the first and second sensor data; generating control signals for varying a valve control system based on the detected changes in the at least one of chlorine concentration, temperature, or pressure of water in the water distribution system; and transmitting, to the valve control system, the control signals.

Other example embodiments of the present disclosure will be apparent to those of ordinary skill in the art from a review of the following detailed descriptions in conjunction with the drawings.

In the present application, the term “and/or” is intended to cover all possible combinations and sub-combinations of the listed elements, including any one of the listed elements alone, any sub-combination, or all of the elements, and without necessarily excluding additional elements.

In the present application, the phrase “at least one of . . . or . . . ” is intended to cover any one or more of the listed elements, including any one of the listed elements alone, any sub-combination, or all of the elements, without necessarily excluding any additional elements, and without necessarily requiring all of the elements.

The COVID-19 pandemic has renewed the interest in water quality monitoring systems for buildings. A utility that supplies water to a property/building is responsible for the quality of water up to the property line. The utility may be generally equipped with sophisticated equipment and trained staff in order to comply with water quality regulations, but these resources are not usually brought to bear within private water systems connected to the public water supply. Large buildings typically have building automation systems (BAS) that control and monitor HVAC, lighting, power, security, fire systems, etc., but such BAS do not include monitoring and control of water systems.

Property managers may encounter water quality issues when they re-open their buildings after long periods of stagnation and reduced water use. Disinfectant concentrations are critical for inhibiting regrowth in pipes, especially if pipe colonization leads to growth of pathogenic microbes. Low occupancy of buildings results in higher water age, lower chlorine levels, and conditions more favorable for biological growth in the water distribution system. Water use patterns may have changed and even the utility supplying water may have difficulty managing water age at times. Under such conditions, maintaining disinfectant residuals in the water supply system for the buildings may be challenging. Disinfectant decay can be especially problematic in buildings that are only partially occupied, or in buildings that are in areas where water use has dropped overall.

In order for a property manager to monitor and maintain disinfectant concentrations in the water distribution system of a typical industrial, commercial, or institutional (ICI) property, they may need access to some or all of the following information: influent chlorine concentration; chlorine concentration at distal areas or areas of high risk of the water distribution system; chlorine demand; water temperature (influent and distal ends); water pressure (influent and distal ends); and amount of flushing required.

Existing devices for measuring chlorine levels range from water quality testing kits for pool maintenance to sophisticated chlorine monitors for use in water treatment facilities. The pool testing kits are inexpensive and easily purchased, but are less accurate and laborious to use. The more sophisticated electronic monitoring devices are generally reliable, accurate and robust; however, these devices tend to have industry-specific connectivity limitations that make access to data inconvenient. There is an unfilled gap in the market for water quality monitoring systems between low-cost, low accuracy testing for pool maintenance and high-cost, high quality monitoring for water treatment plants.

The present application discloses a system for monitoring the quality of water in a water distribution system of a building. The system employs a plurality of sensor assemblies that are disposed at different points along the water distribution system to collect sensor data. Based on the sensor data, the system is operable to, for example, detect changes in water quality conditions, provide suggested actions for addressing water quality issues, and notify users when certain defined alarm (or trigger) conditions are detected. The system may assist users in evaluating one or more of the following: sufficiency of chlorine concentration in municipal influent; sufficiency of chlorine concentration in building water distribution; requirement for water flushing; requirement for chlorine addition; effectiveness of flushing practices for the building; and impact of flushing or other actions on water pressure.

A well-operated water distribution system for a building or property may provide, at least, consistent chlorine concentration, cold water temperatures below 20° C., hot water temperatures above 50° C., and consistent water pressures such that backflow or contamination events are not likely. Low pressure issues are likely to be found upstream of booster pumps, in the area of flushing valves, at higher elevations, or downstream of major pipe constrictions. With suitably-placed sensor assemblies, the chlorine, temperature, and pressure data from the water quality monitoring system may be used to address issues of low chlorine, high or low water temperature, or low pressure. Additional data from other types of sensors, such as biofilm sensors, may add validity and more direct indications of water quality issues. The data may be used for triggering certain automatic or manual actions such as, for example, flushing, chlorine addition, modification of plumbing system, modification of flushing protocols, addition of booster pumps, insulation of pipes, and modification of water usage patterns.

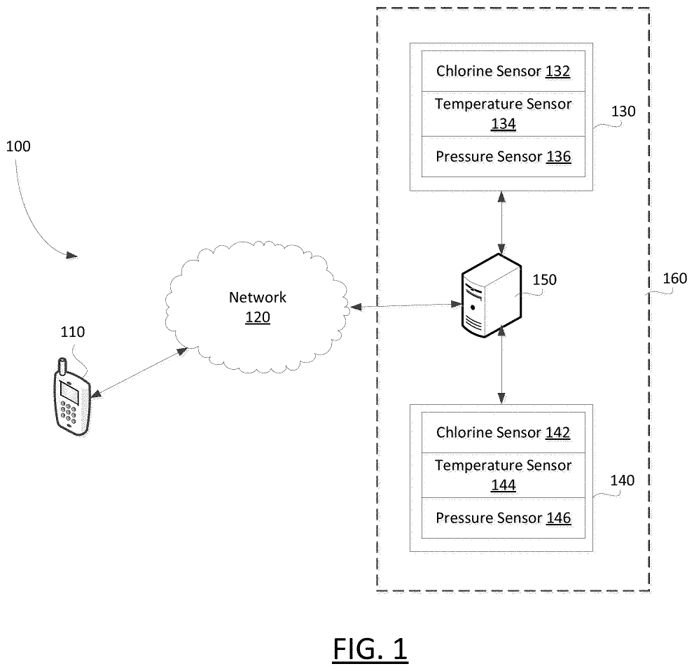

Reference is first made to FIG. 1, which is a schematic diagram illustrating an operating environment of an example embodiment. Specifically, FIG. 1 illustrates exemplary components of a system 100 for monitoring the quality of water in a water distribution system of a building. The system 100 may be suitable for implementing in various different types of facilities, such as residential buildings, commercial office and government buildings, shopping malls, museums, libraries, university campus buildings, and the like. While the present disclosure describes embodiments of water quality control systems for individual buildings, it will be understood that the technology disclosed herein may be implemented more generally for controlling water quality across multiple buildings of a property.

The system 100 includes a plurality of sensor assemblies. FIG. 1 illustrates a first sensor assembly 130 and a second sensor assembly 140. The sensor assemblies 130 and 140 are used for sampling water and collecting sensor data. Specifically, the sensors of the sensor assemblies 130 and 140 are used to obtain measurements of water quality parameters which may include, among others, chlorine concentration, temperature, pressure, pH, dissolved oxygen (DO) concentration, redox potential (ORP), turbidity, salinity, and stream flow. The first sensor assembly 130 includes at least one of: a chlorine sensor 132 for detecting a concentration of free or total chlorine in water, a temperature sensor 134, or a pressure sensor 136. The second sensor assembly 140 at least one of: a chlorine sensor 142, a temperatures sensor 144, or a pressure sensor 146. The chlorine sensor is an example of a disinfection sensor; in some embodiments, the first and second sensor assemblies may include a different disinfection sensor (e.g., hydrogen peroxide sensor) in addition to or in place of a chlorine sensor. Other types of sensors may be suitable for inclusion in the first and second sensor assemblies. For example, in some embodiments, each of the first and second sensor assemblies may additionally include at least one of: flow control valves; pressure regulator valves; a variable-area flowmeter; a turbidimeter; an adenosine triphosphate (ATP) analyzer; bioelectrode sensors; flow cytometry equipment; or one or more drain pipes.

The sensor assemblies 130 and 140 are installed at specific locations in the building to facilitate collection of sensor data at different points along the water distribution system. Specifically, the first sensor assembly 130 may be installed at a location on the property that is as close as possible to where the water supply enters the property. This location may, for example, be in a basement or utility room that provides direct access to influent water (e.g., a ½″ or ¼″ sample tap) and drain or sanitary connection for wastewater. More generally, the first sensor assembly 130 is installed to be fluidly connected to the water distribution system at a first connection point such that influent water from a water treatment system (or other external water source) flows at least partially through the first sensor assembly 130. The first sensor assembly 130 is adapted to collect samples of the influent water that flows through the first sensor assembly 130.

The second sensor assembly 140 is disposed at a location that is downstream of the first sensor assembly 130 in the water distribution system. Specifically, one or more second sensor assemblies 140 may be fluidly connected to the water distribution system at second connection points that are downstream of the first connection point. For example, the second sensor assemblies 140 may be located at one or more distal ends of the water distribution system. The number and locations of the second sensor assemblies will depend on factors such as size, complexity, water uses, etc. of the water distribution system. The second sensor assemblies 140 are adapted to collect samples of the water that flows through the second sensor assemblies 140.

The sensors of the first sensor assembly 130 and second sensor assembly 140 are installed so as to be exposed to flowing water from the water distribution system. This may be achieved by, for example, diverting a portion of the property's water flow via tubing to the sensors housed in the sensor assemblies 130 and 140.

The sensor assemblies 130 and 140 may be configured to store measurements of various water quality parameters. For example, each sensor of the sensor assemblies 130 and 140 may include an internal memory which contains a database storing measurements that are obtained using the sensor. Additionally, or alternatively, each sensor may be communicably connected to a controller, a remote database, etc. that is adapted to receive and store sensor measurements from the sensors.

The system 100 also includes a controller 150. The controller 150 is configured to poll for and collect measurement data from the sensors of the sensor assemblies 130 and 140. In at least some embodiments, the controller 150 may administer, monitor, and/or control the sensors, and the sensors may be configured to receive and execute commands transmitted by the controller 150. For example, the controller 150 may be enabled to, for each of the one or more sensors, configure and update device settings over-the-air, enforce compliance policies, and/or remotely deploy instructions.

The controller 150 may be implemented as a stand-alone device that is connected to the network 120 or as part of a remote server, such as a collection of one or more server computers. In some embodiments, the controller 150 may be configured to receive input via one or more input interfaces. For example, the controller 150 may include a plurality of control buttons, a keypad, and/or a touch-sensitive overlay associated with a touchscreen display. The controller 150 may also be configured to send and receive communication from building automation and control systems, using communications protocols such as BACnet, Modbus, or LonWorks.

A building may be provided with a single controller 150 or multiple controllers 150. For example, a building may have a main controller coupled to a sub-network of controllers, where the sub-network may comprise one or more controllers for each of a plurality of floors/levels, zones, etc. of the building. Alternatively, in some embodiments, the building may have a plurality of controllers 150, each of which is communicably connected to a single main backend server.

The first sensor assembly 130, the second sensor assembly 140, and the controller 150 are components of a water quality monitoring system 160 for a building. In the example embodiment illustrated in FIG. 1, the sensor assemblies 130 and 140 are communicably coupled to the controller 150 which is, in turn, connected to the network 120. In some embodiments, the water quality monitoring system 160 may include additional components, such as a valve control system or chlorine injection pumps, that facilitate controlling the quality of water in the water distribution system of the building.

FIG. 1 also illustrates a client device 110. The client device 110 is a computing device. For example, the client device 110 may be a device of an entity (e.g., a property manager) that is associated with the water distribution system of the building. The client device 110 may take a variety of forms including, for example, a mobile communication device such as a smartphone, a tablet computer, a wearable computer such as a head-mounted display or smartwatch, a laptop or desktop computer, or a computing device of another type.

As illustrated, the client device 110, the sensor assemblies 130 and 140, and the controller 150 communicate via the network 120. The network 120 is a computer network. In some embodiments, the network 120 may be an internetwork such as may be formed of one or more interconnected computer networks. For example, the network 120 may be or may include an Ethernet network, an asynchronous transfer mode (ATM) network, a wireless network, or the like.

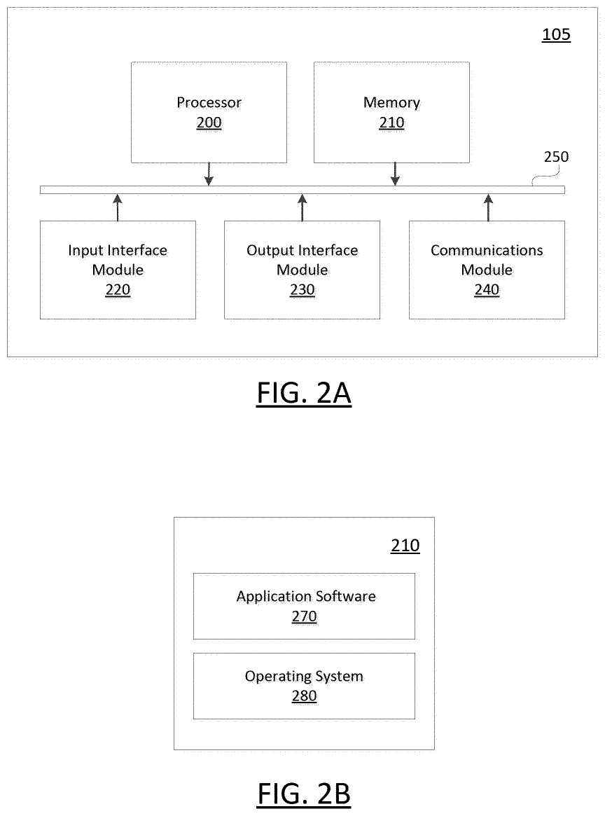

FIG. 2A is a high-level operation diagram of an example computing device 105. In at least some embodiments, the example computing device 105 may be exemplary of one or more of the client device 110, sensors of the sensor assemblies 130 and 140, and the controller 150. The example computing device 105 includes a variety of modules. For example, the example computing device 105, may include a processor 200, a memory 210, an input interface module 220, an output interface module 230, and a communications module 240. As illustrated, the foregoing example modules of the example computing device 105 are in communication over a bus 250.

The processor 200 is a hardware processor. For example, the processor 200 may be one or more ARM, Intel x86, PowerPC processors or the like.

The memory 210 allows data to be stored and retrieved. The memory 210 may include, for example, random access memory, read-only memory, and persistent storage. Persistent storage may be, for example, flash memory, a solid-state drive or the like. Read-only memory and persistent storage are a computer-readable medium. A computer-readable medium may be organized using a file system such as may be administered by an operating system governing overall operation of the example computing device 105.

The input interface module 220 allows the example computing device 105 to receive input signals. Input signals may, for example, correspond to input received from a user. The input interface module 220 may serve to interconnect the example computing device 105 with one or more input devices. Input signals may be received from input devices by the input interface module 220. Input devices may, for example, include one or more of a touchscreen input, keyboard, trackball or the like. In some embodiments, all or a portion of the input interface module 220 may be integrated with an input device. For example, the input interface module 220 may be integrated with one of the aforementioned example input devices.

The output interface module 230 allows the example computing device 105 to provide output signals. Some output signals may, for example allow provision of output to a user. The output interface module 230 may serve to interconnect the example computing device 105 with one or more output devices. Output signals may be sent to output devices by output interface module 230. Output devices may include, for example, a display screen such as, for example, a liquid crystal display (LCD), a touchscreen display. Additionally, or alternatively, output devices may include devices other than screens such as, for example, a speaker, indicator lamps (such as for, example, light-emitting diodes (LEDs)), and printers. In some embodiments, all or a portion of the output interface module 230 may be integrated with an output device. For example, the output interface module 230 may be integrated with one of the aforementioned example output devices.

The communications module 240 allows the example computing device 105 to communicate with other electronic devices and/or various communications networks. For example, the communications module 240 may allow the example computing device 105 to send or receive communications signals. Communications signals may be sent or received according to one or more protocols or according to one or more standards. For example, the communications module 240 may allow the example computing device 105 to communicate via a cellular data network, such as for example, according to one or more standards such as, for example, Global System for Mobile Communications (GSM), Code Division Multiple Access (CDMA), Evolution Data Optimized (EVDO), Long-term Evolution (LTE) or the like. Additionally, or alternatively, the communications module 240 may allow the example computing device 105 to communicate using near-field communication (NFC), via Wi-Fi™, using Bluetooth™ or via some combination of one or more networks or protocols. In some embodiments, all or a portion of the communications module 240 may be integrated into a component of the example computing device 105. For example, the communications module may be integrated into a communications chipset.

Software comprising instructions is executed by the processor 200 from a computer-readable medium. For example, software may be loaded into random-access memory from persistent storage of memory 210. Additionally, or alternatively, instructions may be executed by the processor 200 directly from read-only memory of memory 210.

FIG. 2B depicts a simplified organization of software components stored in memory 210 of the example computing device 105. As illustrated, these software components include application software 270 and an operating system 280.

The application software 270 adapts the example computing device 105, in combination with the operating system 280, to operate as a device performing a particular function. For example, when the client device 110 is adapted as the computing device 105, the application software 270 may include a water quality monitor application. The water quality monitor application may enable users to perform various actions using the client device 110 such as, but not limited to: configuring network connections for the sensor assemblies; calibrating the sensors of the sensor assemblies; labelling the sensor assemblies; inputting system information such as flow rates, pipe diameters, distance between monitoring stations, etc.; configuring alarm notifications and viewing alarm histories; real-time monitoring of chlorine, pressure, and temperature data; receiving alarms and/or notifications for water quality issues; receiving indications of suggested actions for responding to alarm conditions.

The operating system 280 is software. The operating system 280 allows the application software 270 to access the processor 200, the memory 210, the input interface module 220, the output interface module 230 and the communications module 240. The operating system 280 may be, for example, Apple iOS™, Google Android™, Linux™, Microsoft Windows™, or the like.

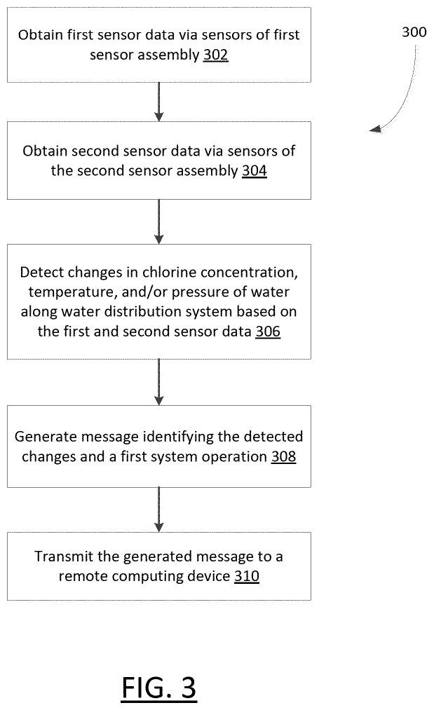

Reference is now made to FIG. 3 which shows, in flowchart form, an example method 300 for operating a water quality monitoring system of a building. The method 300 may be implemented by a controller associated with a water quality monitoring system, such as the controller 150 of FIG. 1. In particular, a computing system that is communicably coupled with and configured to control one or more components of a water quality monitoring system may implement the method 300. Operations starting with operation 302 and continuing onward may be performed, for example, by the processor 200 (FIG. 2A) of a computing device 105 executing software comprising instructions such as may be stored in the memory 210 of the computing device 105. Specifically, processor-executable instructions may, when executed, configure a processor 200 of a controller 150 to perform all or parts of the method 300.

In operation 302, the controller obtains first sensor data via sensors of the first sensor assembly, such as the first sensor assembly 130 of FIG. 1. In particular, the controller may obtain, at a first time, sensor data indicating chlorine concentration, temperature, and pressure of water via the sensors of the first sensor assembly. The first sensor assembly is fluidly connected to the water distribution system at a first connection point such that influent water from an external water source (e.g., municipal water treatment plant) flows at least partially through exposed sensors of the first sensor assembly. The controller may, for example, poll the sensors of the first sensor assembly at defined times and/or periodically, or on command (e.g., in response to a user-initiated request to obtain the first sensor data). The obtained first sensor data may be associated with the first time. For example, the first sensor data may be stored in a memory in association with a timestamp indicating the first time.

In some embodiments, the first connection point may be physically located in the basement or utility room of a building. In particular, if there is a single inlet to a building's water distribution system, only one first connection point may be defined for the building. On the other hand, if there are additional defined inlets, such as for larger water users (e.g., a university campus) that have multiple connections to a municipal water supply system, a plurality of first connection points may be identified as locations for one or more first sensor assemblies.

In operation 304, the controller obtains second sensor data via sensors of the second sensor assembly, such as the second sensor assembly 140 of FIG. 1. Specifically, the second sensor data may be obtained at second times that are after the first time. The second times may, for example, be a predefined length of time after the first time. A water quality monitoring system may include a plurality of second sensor assemblies that are located at different points throughout a building. In particular, one or more second sensor assemblies are disposed downstream of the first sensor assembly in the water distribution system at second connection points. In at least some embodiments, the second sensor assemblies are located at distal ends (e.g., top of office tower, building wings that are furthest from a utility connection point, etc.) or high-risk areas (e.g., showers, cooling towers, etc.) of the water distribution system. The second sensor data indicate values of, at least, chlorine concentration, temperature, and pressure of water that is sampled at the second connection points. The obtained second sensor data from a second sensor assembly may be associated with a respective second time. For example, the second sensor data may be stored in a memory in association with a timestamp indicating the respective second time.

Sensor assembly locations downstream of the first sensor assembly may be determined based on risk. Various different high-risk locations may be identified within a building or property. In some embodiments, sensor assemblies may be located in areas where chlorine concentrations are likely to be low (e.g., areas furthest from the inlet(s)) and/or where consequence of regrowth may be severe (i.e., where water is likely to be aerosolized and where at-risk people are likely to be exposed). Locations with low chlorine concentrations may include, but are not limited to: areas furthest from the inlet(s) where water age is the greatest (e.g., upper floors of a building, on opposite side of building from inlet); areas with lowered water use despite having larger diameter water pipes (e.g., lowered occupancy areas of building); areas with frequent stagnation, allowing water temperature in cold water pipes to rise (e.g., seasonal or weekly stagnations); areas where cold water is often warmed to between 20 and 50° C.; and areas where old pipes have high surface areas or sediment.

Additionally, or alternatively, second sensor assemblies may be installed in locations within a building or property where airborne pathogens are highly likely to be found (i.e., high-risk for water spray). Such locations may include, among others: wash systems such as for dishes and food processing; vegetable and fruit misters in grocery stores; showers; cooling misters on patios; water fountains; cooling towers; and sprinkler systems for irrigation.

Depending on what sensors are incorporated in the second sensor assemblies, additional areas of a building/property may be considered for second sensor assembly installation. In some cases, temperature may be an indicator of changes in water conditions that favor growth of pathogens. In other cases, additional monitoring technologies, such as biofilm monitoring, may be used to detect actual regrowth. Direct measurements of regrowth may provide good indication of potential risk of outbreaks.

Stagnating cold water pipes, especially in areas exposed to warm temperatures (e.g., between 20 and 50° C.) may create an ideal environment for Legionella growth, if the higher water temperature aids in decay of chlorine. As another example, because high temperature water is associated with more rapid chlorine decay, hot water systems typically have low chlorine concentrations. If recirculation or stagnation allows the water to remain below 50° C., Legionella growth may be supported. As yet further examples, areas where pipe configurations promote sedimentation or scale growth or where pipe roughness supports regrowth may be suitable locations for installing second sensor assemblies.

The controller may be configured to identify locations of high risk based on, for example, user input including definition of the locations and/or sensor measurements from sensors located throughout the building or property. The second sensor assemblies may then be installed at one or more of the identified locations. In some embodiments, at least one of the second sensor assemblies may be located downstream of flushing valves that are used for releasing volumes of water when activated. That is, one or more second sensor assemblies may be disposed in the water distribution system such that sensors of said second sensor assemblies are configured to measure water quality parameters after controlled flushing of water occurs. In this way, the effects of flushing on the water quality parameters may be monitored based on differences in measurements at the first and second sensor assemblies.

In at least some embodiments, the controller may be configured to continuously monitor water quality parameters based on data obtained via sensors of the sensor assemblies. In particular, the controller may maintain, at least, chlorine concentration, temperature, and pressure data for sampled water on an ongoing basis, without receiving express requests to determine water quality indicators.

When attempting to poll sensors to obtain water quality parameter data, one or more of the sensors may not be properly configured or connected to the controller. In some embodiments, if a sensor is not responsive to a polling request from the controller, an alert notification can be generated to identify a polling failure. For example, if communication with a sensor cannot be established or is lost after having been established, multiple attempts can be made to re-establish connection to the sensor. If, after a predetermined number of attempts, communication with the sensor is unsuccessful, a notification message indicating communication failure may be generated and transmitted to a backend server or administrator.

In operation 306, the controller detects changes in at least one of chlorine concentration, temperature, or pressure of water between the first connection point and the one or more second connection points based on the first and second sensor data. For example, the controller may determine an amount of change in chlorine concentration, temperature, and/or pressure of water between the first and second connection points, and compare the amount of change to one or more relevant threshold values defined for the respective parameter. If the amount of change for a parameter exceeds a respective threshold value, the controller may determine that a substantial change in the parameter has occurred. Additionally, or alternatively, the controller may compare values from both the first sensor assembly 130 and the second sensor assemblies 140 for at least one of chlorine concentration, temperature, or pressure of water against defined threshold values. If a value of a parameter for any one of the first sensor assembly 130 and the second sensor assemblies 140 exceeds a defined threshold value, the controller may determine that a condition exists that requires a response.

The first and second sensor data may inform determination of additional indicators of water quality such as, but not limited to, chlorine decay, water age in water distribution system, and chlorine decay rate. In some embodiments, the controller may generate historical trends data based on sensor data obtained via sensors of the first and second sensor assemblies. The first and second sensor data may be used in conjunction with historical trends data in determining flushing parameters for the water distribution system such as: flushing time required to replace one volume of system water; flushing time required to re-establish desired chlorine concentration at one or more of the sensor assemblies; flushing volume required; and cost of flushing.

In some embodiments, the historical trends data may indicate changes in one or more of the water quality parameters (e.g., chlorine concentration, temperature, pressure, etc.) from the first sensor assembly through one or more second sensor assemblies. The value of a water quality parameter may change over time as water passes from the influent (first) sensor assembly to distal (second) sensor assemblies. The historical trends data may indicate the variation of a water quality parameter over time, which may assist in evaluating the amount of time it takes for changes in chlorine level, temperature, etc. to pass through the water distribution system. For example, based on how long changes in temperature and/or chlorine level require to pass from the influent sensor assembly to distal assemblies during normal periods of water use, the controller may be configured to determine an estimate of the duration that water resides in the water distribution system (i.e., water age).

If the detected changes in measured sensor values are determined to satisfy certain conditions, the controller generates a message indicating such changes, for transmission to one or more suitably identified computing devices. Specifically, responsive to determining that the detected changes satisfy defined criteria, the controller generates a message identifying the detected changes and at least a first system operation for performing in connection with the water quality monitoring system, in operation 308.

In at least some embodiments, the generated message may include control signals for varying a valve control system based on the detected changes in at least one of chlorine concentration, temperature, or pressure of water in the water distribution system. The valve control system may be operable to control valves of the water distribution system. In particular, the valves may be controlled for flushing water from pipes of the water distribution system. In some embodiments, the control signals may be generated based on both the detected changes and historical trends data associated with the sensor data from the first and second sensor assemblies. The control signals may include instructions for causing an automated control valve actuator to open and close valve(s) such that certain water pipes in the property water system are flushed (e.g., to achieve a desired chlorine residual, flush a volume of water, achieve a certain temperature, etc.). Depending on the required type of signal, the output may be: a simple 24 VAC electrical signal to operate a valve (typical of simple irrigation valves), or a 4-20 mA signal or other signal to a water system programmable logic controller (PLC) that controls valve actuators. This PLC could be integrated into the monitoring system or could be part of a separate system.

In some embodiments, if the water quality monitoring system is used in conjunction with a chlorine booster system or other chlorination system, the water quality monitoring system may output signals based on a desired chlorine residual setpoint to operate said disinfection system such that injection pumps operate to increase or restore a disinfectant concentration in the property water system to a desired concentration. The water quality monitoring system may be used to send electrical signals directly to a pump to cause it to inject disinfectant, or the water quality monitoring system may relay signals to a PLC that operates the disinfectant pump. The operation of the pump may be modulated such that the speed of the pump is proportional to the amount of disinfectant required; if the actual chlorine concentration is only slightly below the desired disinfectant concentration, only a small amount of additional disinfectant may be required.

The controller transmits the message, such as a message including control signals for a valve control system, to a remote computing device in operation 310. The remote computing device may, for example, be a device associated with an operator of the water quality monitoring system or a remote computer server that is configured to adjust one or more control variables of the water quality monitoring system.

Various different types of outputs may be provided by the water quality monitoring system. In some embodiments, the water quality monitoring system may provide indication of sensor assembly status of one or more of the first and second sensor assemblies. A sensor assembly may present, via an LED or device-mounted screen, information such as: red flashing LED for alarm conditions (e.g., low chlorine, maintenance required, or other issue); green light for a ‘clear’ operating status; and display of current sensor measurement data (e.g., chlorine, pressure, temperature, etc.). The water quality monitoring system may provide indication of sensor assembly information to one or more connected devices, by transmitting relevant information such as operating status, current sensor readings, previous calibration(s), and maintenance history to those devices for display thereon.

In some embodiments, the water quality monitoring system may output notifications (e.g., via email, in-app notification, etc.) to communicate information such as: device status (e.g., scheduled maintenance required, current sensor assembly readings, maintenance history); system configuration details (sensor assembly location details, pipe diameters and lengths upstream of sensor assemblies, operating threshold values, alarm setpoints, alarm actions); sensor readings detected outside of threshold settings; and messages conveying suggested actions to improve water quality conditions.

The detected changes in chlorine concentration, temperature, and/or pressure of water based on the first and second sensor data may be used for generating signals to control other components of the water distribution system. In some embodiments, the controller may generate control signals for operating a chlorine injection pump to control a chlorine flow rate into the water distribution system. For example, in response to determining that a decrease in chlorine concentration between the first and second sensor assemblies exceeds a defined threshold amount, the controller may generate control signals for increasing a flow rate of a chlorine injection pump that is fluidly connected to the water distribution system.

The controller may reset and/or calibrate each sensor of the sensor assemblies separately or reset sensors in groups (e.g., reset all sensors on a floor level, etc.). If some sensors do not respond when a reset is requested, the controller may attempt to reset the sensors multiple times, with predetermined delay periods (e.g., two second delay) between the reset requests.

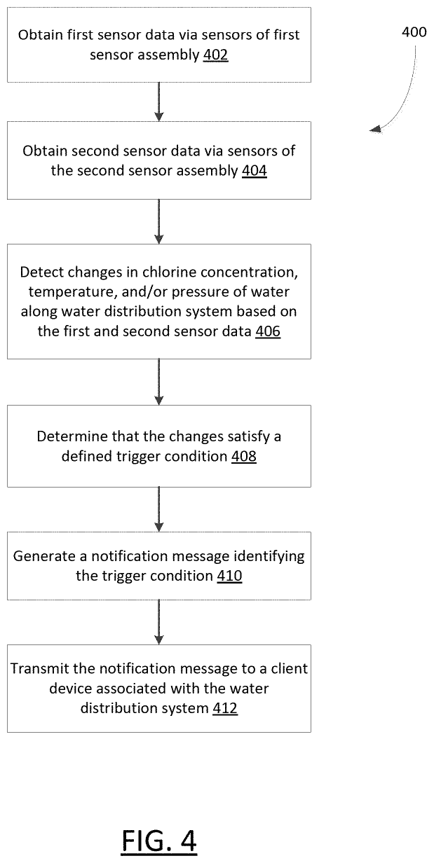

Reference is now made to FIG. 4 which shows, in flowchart form, another example method 400 for operating a water quality monitoring system of a building. The method 400 may be implemented by a controller associated with a water quality monitoring system, such as the controller 150 of FIG. 1. In particular, a computing system that is communicably coupled with and configured to control one or more components of a water quality monitoring system may implement the method 400. Operations starting with operation 402 and continuing onward may be performed, for example, by the processor 200 (FIG. 2A) of a computing device 105 executing software comprising instructions such as may be stored in the memory 210 of the computing device 105. Specifically, processor-executable instructions may, when executed, configure a processor 200 of a controller 150 to perform all or parts of the method 400. The operations of method 400 may be performed in addition to, or as alternatives, of one or more operations of method 300.

In operation 402, the controller obtains first sensor data via sensors of a first sensor assembly at a first time. The first sensor assembly is located in the building such that it is fluidly connected to the water distribution system at a first connection point allowing influent water from an external water source to flow at least partially through exposed sensors of the first sensor assembly. The first sensor assembly includes, at least, a chlorine sensor for detecting a concentration of chlorine in water, a temperature sensor, and a pressure sensor. The controller may poll the sensors of the first sensor assembly at defined times, periodically, or in response to commands (i.e., user-initiated requests).

In operation 404, the controller obtains second sensor data via sensors of one or more second sensor assemblies at second times that are after the first time. The second sensor assemblies are disposed downstream of the first sensor assembly and are located at one or more second connection points along the water distribution system of the building. For example, the second sensor assemblies may be installed at one or more distal ends or high-risk areas of the water distribution system. The controller may poll the sensors of the second sensor assemblies at defined times, periodically, or in response to commands (i.e., user-initiated requests).

The controller detects changes in chlorine concentration, temperature, and/or pressure of water along the water distribution system between the first and second connection points based on the first and second sensor data, in operation 406. For example, the controller may determine that measured values of water quality parameters have increased or decreased at different connection points. The controller may be configured to determine whether said changes are significant changes which may affect water quality. In particular, the controller may check whether the first sensor data, the second sensor data, and/or the changes in the sensor data satisfy one or more defined trigger conditions, in operation 408.

In some embodiments, the trigger condition(s) may be associated with a threshold value, and the controller may be configured to compare the first and second sensor data (or changes therebetween) with the threshold value. For example, a trigger condition may be determined to be satisfied if the difference in a parameter (e.g., chlorine concentration) between the first and second sensor data exceeds a defined threshold value. As another example, a trigger condition may be determined to be satisfied if the difference in a parameter is equal to or less than a defined threshold value. As yet another example, a trigger condition may be determined to be satisfied if a value of a parameter measured by a sensor of any one of the sensor assemblies exceeds (or is less than) a defined value at certain time(s).

In response to determining that the first sensor data, the second sensor data, and/or the changes in the sensor data satisfy defined trigger conditions, the controller generates a notification message identifying the trigger condition (operation 410) and transmits the notification message to a client device associated with the water distribution system (operation 412). For example, the notification message may include an indication of a parameter (e.g., chlorine concentration), a defined threshold value associated with the parameter, and one or more suggested actions for handling a water quality issue associated with exceeding the threshold value.

Additionally, various different types of notification messages may be generated and provided to client devices associated with the water distribution system. In some embodiments, the controller may be configured to obtain current and/or historical sensor data associated with sensors of one or more other related buildings, and generate notifications based on said sensor data. For example, the controller may aggregate and compare sensor data from other facilities in a same or nearby geographical region and determine whether any one or more of the facilities is experiencing issues with water quality. The other facilities may be similarly outfitted with the sensor assemblies that are described in the present application. In particular, the controllers associated with sensor assemblies of different facilities may be configured to communicate with each other to share sensor data and/or indications of detected water quality issues or conditions.

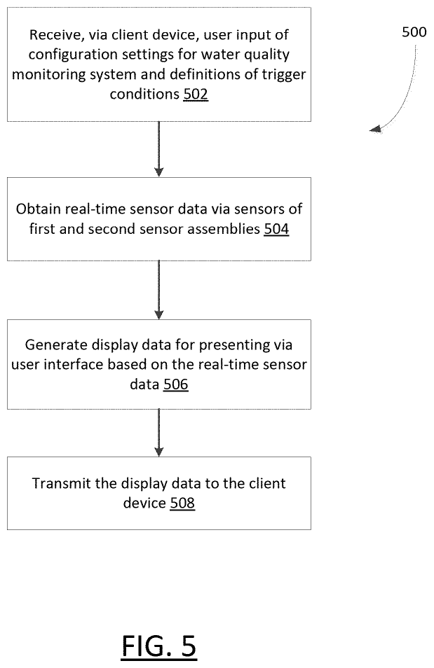

Reference is now made to FIG. 5 which shows, in flowchart form, an example method 500 for providing, on a client device, a user interface for controlling a water quality monitoring system. The method 500 may be implemented by a controller associated with a water quality monitoring system, such as the controller 150 of FIG. 1. In particular, a computing system that is communicably coupled with and configured to control one or more components of a water quality monitoring system may implement the method 500. Operations starting with operation 502 and continuing onward may be performed, for example, by the processor 200 (FIG. 2A) of a computing device 105 executing software comprising instructions such as may be stored in the memory 210 of the computing device 105. Specifically, processor-executable instructions may, when executed, configure a processor 200 of a controller 150 to perform all or parts of the method 500. The operations of method 500 may be performed in addition to, or as alternatives, of one or more operations of methods 300 and 400.

In operation 502, the controller receives, via a client device, user input of configuration settings for the water quality monitoring system and definitions of one or more trigger conditions. A user of the client device may input, using a mobile app (or other application software), the configuration settings. For example, the user may select from one or more predefined settings and/or trigger conditions, or manually input definitions (e.g., parameters, threshold values, etc.) of desired alarm conditions.

In operation 504, the controller obtains real-time sensor data via sensors of first and second sensor assemblies, such as the assemblies 130 and 140 of FIG. 1. In particular, the controller may continuously poll the sensors to obtain measurements of water quality parameters.

In operation 506, the controller generates display data for presenting via a user interface based on the real-time sensor data. In some embodiments, the display data may include graphical representations of sensor data associated with the first and second sensor assemblies.

The controller then transmits the display data to the client device, in operation 508. The display data may be transmitted as push notifications for the client device. More generally, the client device can be used to define alarm setpoints and notifications can be sent to alert user(s) of the client device to specific conditions of concern relating to the quality of water in the water distribution system of the building. Once an alarm is triggered, the user(s) may be notified.

Additionally, the user interface on the client device may be used for delivering indications of suggested actions corresponding to detected alarm/trigger conditions. By way of illustration and not limitation, the following Table 1 outlines various water quality conditions and corresponding actions which may be indicated, via the user interface, by the controller responsive to detecting the conditions. For example, notifications, pop-ups, or other similar messages indicating one or more of the suggested actions may be presented via a graphical user interface.

TABLE 1

| Alarm condition | Suggested action(s) |
|---|---|
| Low chlorine | The low chlorine result may be due to low |
| concentration at | sample flow through the analyzer. The user |
| influent | should check the sample flow rate and correct if |
| | necessary. |
| | The low chlorine result may be due to aging of |
| | the chlorine analyzer itself. Recommended |
| | maintenance schedules should prevent |
| | analyzers operating beyond their service life. If |
| | the analyzer is too old, the user should replace |
| | the analyzer head with a new head, calibrate, |
| | and put back into service. |
| | The user should initiate flushing to get more |
| | fresh water into the property water system. The |
| | user should consider notifying others using the |
| | property water system of potential risks |
| | If flushing does not increase the chlorine |
| | concentration, there may be too large a volume |
| | of poorly disinfected water in the municipal |
| | system to be flushed by the user. The water |
| | supplier should be informed so that hydrant |
| | flushing can be conducted without incurring |
| | flushing costs to the user. |
| Low chlorine | The low chlorine result may be due to low |
| concentration | sample flow through the analyzer. The user |
| at distal | should check the sample flow rate and correct if |
| location(s) | necessary. |
| | The low chlorine result may be due to aging of |
| | the chlorine analyzer itself. Recommended |
| | maintenance schedules should prevent |
| | analyzers operating beyond their service life. If |
| | the analyzer is too old, the user should replace |
| | the analyzer head with a new head, calibrate, |
| | and put back into service. |
| | The user should initiate flushing to get fresher |
| | water to the distal location. The user should |
| | consider notifying others using the property |
| | water system of potential risks |
| | If flushing does not quickly re-establish the |
| | chlorine residual, the user should review flow |
| | rates and activities in the property water system |
| | for possible causes. If a cause is identified, such |
| | as the return of an old water line to service, the |
| | user should review whether continued flushing |
| | can address the low chlorine or if other actions |
| | are required, such as additional chlorination. |
| High water | Conduct flushing at influent or distal locations, |
| temperature in cold | depending on where high temperatures are |
| water piping | detected. |
| | Insulate or re-route pipes where they are |
| | exposed to high ambient temperatures |
| | Proactively add additional chlorine to |
| | compensate for the higher chlorine decay rate at |
| | elevated temperatures |
| | Turn off high-risk water uses if chlorine |
| | concentrations and temperatures cannot be |
| | controlled in non-essential water uses (such as |
| | in decorative fountains) |
| Low water temperature | Increase water temperature in water heater |
| in hot water | Increase flow rates or insulation in hot water |
| piping | piping |
| Low pressure | The user should investigate whether the low |
| | pressure is related to activities such as fire |
| | suppression, flushing or pipe bursts. If flushing |
| | at distal locations is responsible for low |
| | pressures elsewhere in the system, either |
| | booster pumps should be brought into service or |
| | lower flow rates should be used during flushing |
| Variable pressure | To reduce severe pressure variations, the user |
| | may choose to install pressure regulating or |
| | relief valves, water hammer arrestors, booster |
| | pumps etc. to reduce water pressure variations, |
| | thus reducing wear on the system, providing |
| | more consistent water supply, and reducing |
| | opportunity for |
| | backflow/syphoning/contamination/pipe bursts |
| Large decrease in | Flush stagnant water from building water |
| chlorine concentration | system (decrease water age) |
| (high chlorine | Determine whether decrease in water |
| demand) | temperature correlates to high water |
| | temperature or water age |

The various embodiments presented above are merely examples and are in no way meant to limit the scope of this application. Variations of the innovations described herein will be apparent to persons of ordinary skill in the art, such variations being within the intended scope of the present application. In particular, features from one or more of the above-described example embodiments may be selected to create alternative example embodiments including a sub-combination of features which may not be explicitly described above. In addition, features from one or more of the above-described example embodiments may be selected and combined to create alternative example embodiments including a combination of features which may not be explicitly described above. Features suitable for such combinations and sub-combinations would be readily apparent to persons skilled in the art upon review of the present application as a whole. The subject matter described herein and in the recited claims intends to cover and embrace all suitable changes in technology.

1. A system for monitoring water quality in a water distribution system of a building, comprising:
a first sensor assembly and one or more second sensor assemblies, each of the first and second sensor assemblies including:
a chlorine sensor for detecting a concentration of chlorine in water;
a temperature sensor; and
a pressure sensor,
wherein the first sensor assembly is fluidly connected to the water distribution system at a first connection point such that influent water from a water treatment system flows at least partially through the first sensor assembly and the one or more second sensor assemblies are disposed downstream of the first sensor assembly in the water distribution system at one or more second connection points;
a processor; and
a memory coupled to the processor, the memory storing computer-executable instructions that, when executed by the processor, cause the processor to:
obtain first sensor data via sensors of the first sensor assembly;
obtain second sensor data via sensors of the one or more second sensor assemblies;
detect changes in at least one of chlorine concentration, temperature, or pressure of water between the first connection point and the one or more second connection points based on the first and second sensor data;
responsive to determining that the detected changes satisfy defined criteria, generate a message identifying the detected changes and a first system operation for performing in connection with the system; and
transmit the generated message to a remote computing device associated with the system.

2. The system of claim 1, wherein the remote computing device comprises a valve control system that is operable to control valves for flushing water from pipes of the water distribution system and wherein the message includes control signals for varying a valve control system based on the detected changes in the at least one of chlorine concentration, temperature, or pressure of water in the water distribution system.

3. The system of claim 1, wherein each of the first and second sensor assemblies comprises at least one of:
flow control valves;
pressure regulator valves;
a variable-area flowmeter;
a turbidimeter; or
one or more drain pipes.

4. The system of claim 1, wherein the one or more second sensor assemblies are disposed at one or more distal locations along the water distribution system.

5. The system of claim 1, wherein detecting the changes comprises:
determining an amount of change in the at least one of chlorine concentration, temperature, or pressure of water between the first and second connection points; and
comparing the amount of change to one or more defined threshold values.

6. The system of claim 1, wherein the computer-executable instructions, when executed by the processor, further cause the processor to generate historical trends data based on sensor data obtained via sensors of the first and second sensor assemblies, and wherein the control signals are generated based on the detected changes and the historical trends data.

7. The system of claim 1, wherein the computer-executable instructions, when executed by the processor, further cause the processor to:
determine that at least one of the first sensor data or the second sensor data satisfies a defined trigger condition; and
in response to the determining:
generate a notification message identifying the trigger condition; and
transmit the notification message to a client device associated with the water distribution system.

8. The system of claim 7, wherein the trigger condition is associated with a threshold value and wherein the notification message includes an indication of a suggested action for handling a water quality issue associated with the threshold value.

9. The system of claim 7, wherein the computer-executable instructions, when executed by the processor, further cause the processor to receive, via the client device, user input including definitions of the trigger condition.

10. The system of claim 1, wherein the computer-executable instructions, when executed by the processor, further cause the processor to:
generate display data including graphical representation of sensor data associated with the first and second sensor assemblies; and
transmit the display data to a client device associated with the water distribution system.

11. A method for operating a water quality monitoring system of a building, the method including:
connecting a first sensor assembly to a water distribution system of the building at a first connection point such that influent water from a water treatment system flows at least partially through the first sensor assembly;
connecting one or more second sensor assemblies at second connection points that are downstream of the first sensor assembly in the water distribution system;
obtaining, via sensors of the first and second sensor assemblies, first sensor data and second sensor data, respectively;
detecting changes in at least one of chlorine concentration, temperature, or pressure of water between the first connection point and the one or more second connection points based on the first and second sensor data;
responsive to determining that the detected changes satisfy defined criteria, generating a message identifying the detected changes and a first system operation for performing in connection with the water quality monitoring system; and
transmitting the generated message to a remote computing device associated with the water quality monitoring system.

12. The method of claim 11, wherein the remote computing device comprises a valve control system that is operable to control valves for flushing water from pipes of the water distribution system and wherein the message includes control signals for varying a valve control system based on the detected changes in the at least one of chlorine concentration, temperature, or pressure of water in the water distribution system.

13. The method of claim 11, wherein each of the first and second sensor assemblies comprises at least one of:
flow control valves;
pressure regulator valves;
a variable-area flowmeter;
a turbidimeter; or
one or more drain pipes.

14. The method of claim 11, wherein the one or more second sensor assemblies are disposed at one or more distal locations along the water distribution system.

15. The method of claim 11, wherein detecting the changes comprises:
determining an amount of change in the at least one of chlorine concentration, temperature, or pressure of water between the first and second connection points; and
comparing the amount of change to one or more defined threshold values.

16. The method of claim 11, further comprising generating historical trends data based on sensor data obtained via sensors of the first and second sensor assemblies, and wherein the control signals are generated based on the detected changes and the historical trends data.

17. The method of claim 11, further comprising:
determining that at least one of the first sensor data or the second sensor data satisfies a defined trigger condition; and
in response to the determining:
generating a notification message identifying the trigger condition; and
transmitting the notification message to a client device associated with the water distribution system.

18. The method of claim 17, wherein the trigger condition is associated with a threshold value and wherein the notification message includes an indication of a suggested action for handling a water quality issue associated with the threshold value.

19. The method of claim 17, further comprising receiving, via the client device, user input including definitions of the trigger condition.

20. The method of claim 11, further comprising:
generating display data including graphical representation of sensor data associated with the first and second sensor assemblies; and
transmitting the display data to a client device associated with the water distribution system.
